(19) United States
(12) Patent Application Publication
(10) Pub. No.: US 2016/0199049 A1
(43) Pub. Date: Jul. 14, 2016

(54) CLOSURE DEVICES AND RELATED METHODS OF USE

(71) Applicant: Turnkey Healthcare Management, Inc., Laguna Beach, CA (US)

(72) Inventor: Peter van der Sluis, Newport Beach, CA (US)

(73) Assignee: Turnkey Healthcare Management, Inc.

(21) Appl. No.: 14/828,672

(22) Filed: Aug. 18, 2015

Related U.S. Application Data
(60) Provisional application No. 62/038,480, filed on Aug. 18, 2014.

(51) Int. Cl. A61B 17/00

(52) U.S. Cl. CPC A61B 17/0057; A61B 2017/1107

(57) ABSTRACT

A closure device may include a first covering configured to cover a first opening in a first body lumen, and a second covering configured to cover a second opening in a second body lumen. The closure device also may include a tension member coupled to both the first opening and the second opening. The tension member may be reciprocally movable between a collapsed configuration and an expanded configuration, and the tension member may be biased toward the collapsed configuration.

### CROSS-REFERENCE TO RELATED APPLICATION(S)

This patent application claims benefit of priority under 35 U.S.C. §119 to U.S. Provisional Patent Application No. 62/038,480 filed Aug. 18, 2014, the entirety of which is incorporated herein by reference.

### TECHNICAL FIELD

Various embodiments of the present disclosure relate generally to closure devices and related methods of use.

### BACKGROUND

Various medical procedures require that artificial connections be made between body lumens. For example, fistulas/anastomosis or other suitable connections can be made between two body lumens. Once an initial procedure is completed, the fistula/anastomosis or other connection can be closed or otherwise sealed off. Even more generally, various medical procedures require an opening be made in a body lumen or organ, such as a blood vessel, for access to a treatment site or for some other purpose. The opening thereafter may need to be closed.

### SUMMARY

In one aspect, the present disclosure is related to a closure device. The closure device may include a first covering configured to cover a first opening in a first body lumen, and a second covering configured to cover a second opening in a second body lumen. The closure device also may include a tension member coupled to both the first opening and the second opening. The tension member may be reciprocally movable between a collapsed configuration and an expanded configuration, and the tension member may be biased toward the collapsed configuration.

### BRIEF DESCRIPTION OF THE FIGURES

The accompanying drawings, which are incorporated in and constitute a part of this specification, illustrate various exemplary embodiments and together with the description, serve to explain the principles of the disclosed embodiments.

FIG. 1 is a schematic view illustration of a closure device in accordance with an embodiment of the present disclosure.

FIG. 2 is another schematic view illustration of a closure device.

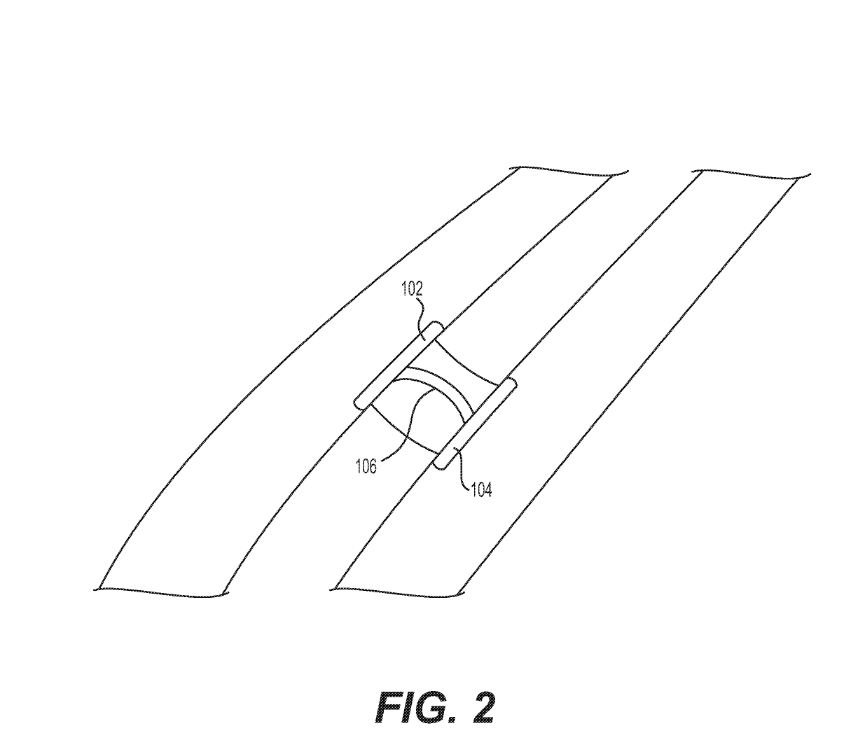

### DETAILED DESCRIPTION

Reference will now be made in detail to embodiments of the present disclosure, examples of which are illustrated in the accompanying drawings. Wherever possible, the same reference numbers will be used throughout the drawings to refer to the same or like parts.

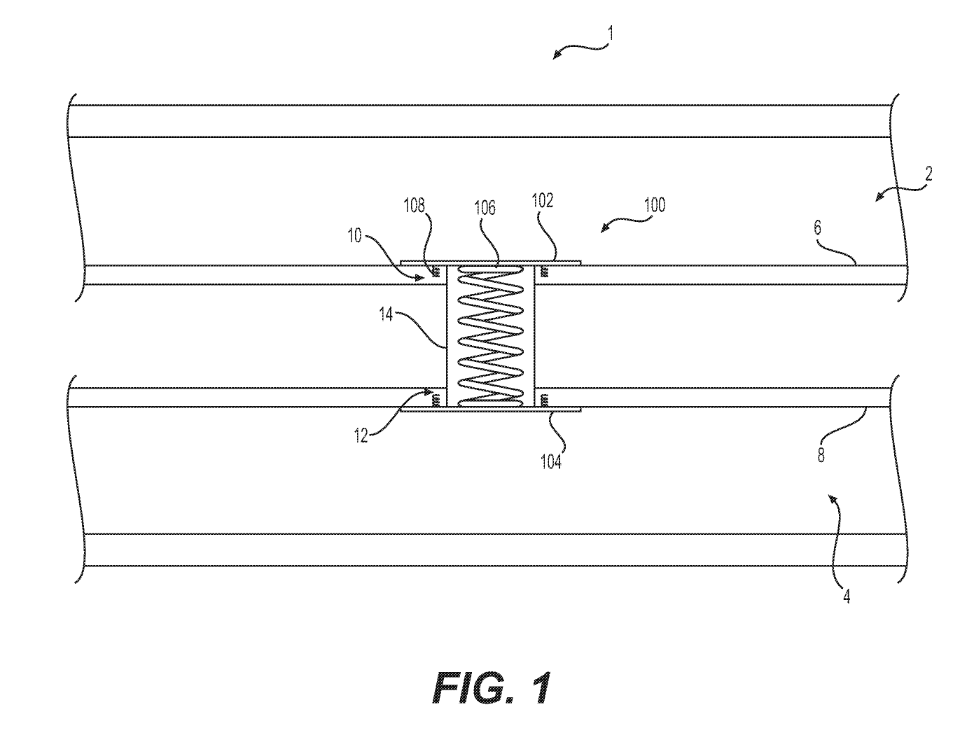

Referring now to the enclosed figures, FIG. 1 is a schematic view illustration of a closure device 100. Closure device 100 may include a first covering 102 and a second covering 104. First and second coverings 102 and 104 may be formed of any suitable biocompatible material. In some examples, first and second coverings 102 and 104 may biodegrade after a suitable amount of time, e.g., days, weeks, months, or years, depending upon the application. In some examples, first and second coverings 102 and 104 may be coated with one or more suitable coatings configured to facilitate regeneration of bodily tissues. First and second coverings 102 and 104 may have a generally large cross-sectional area so as to cover one or more openings formed in the body, but may be of a generally small thickness so as not to disrupt the natural flow of bodily fluids when disposed within a body lumen. In one example, first and second coverings may be formed as discs with a circular, oval, or other like shape. Each of first and second coverings 102 and 104 may be independently collapsible and/or expandable for insertion into a body lumen and through, e.g., a fistula/anastomosis. First and second coverings 102 and 104, or only one of first and second coverings 102, 104, may include one or more attachment features 108 configured to facilitate attachment to bodily tissues. Attachment features 108 may include barbs, hooks, spikes, needles or other features configured to secure first and second coverings 102 and 104 to bodily tissues. In some examples, attachment features 108 may be selectively actuatable by suitable mechanisms to prevent excessive damage to non-targeted bodily tissues during the insertion of closure device 100. In other examples, attachment features 108 may include one or more bio-adhesive coatings configured to couple first and second coverings 102 and 104 to bodily tissues.

First and second coverings 102 and 104 may be coupled to one another by a tension member 106. Tension member 106 may be any suitable tension member, such as, e.g., a spring, coil, compressible polymer, shape memory material, tension arm, or other suitable tension member. In some examples, closure device 100, including its tension member 106, may be reciprocally movable from a collapsed configuration to an expanded configuration. In some examples, closure device 100 and tension member 106 may be biased toward the collapsed configuration, for example a configuration in which tension member 106 has minimal length and first covering 102 is closest to second covering 104. In some examples, one or more of first and second coverings 102, 104 may be independently articulatable with respect to tension member 106.

In one example, one or more of first covering 102, second covering 104, tension member 106, and attachment features 108 may be formed of nitinol or other suitable material, including, but not limited to, stainless steel or other metal alloys, or polymers. Closure device 100 may be implanted by any suitable mechanism, such as, e.g., a percutaneous delivery system.

Closure device 100 may be used in a body system 1. The body system 1 may include a first lumen 2 (e.g., an artery) and a second lumen 4 (e.g., a vein). First lumen 2 may be defined by a first wall 6, while second lumen 4 may be defined by a second wall 8. In some examples, a first opening 10 may be formed in first wall 6, and a second opening 12 may be formed in second wall 8. A fistula/anastomosis 14 or other suitable connection may be formed through first and second openings 10 and 12, and tissues there between, to couple first and second lumens 2 and 4 to one another. Fistula/anastomosis 14 may be a medically implanted device that, e.g., allows a medical practitioner to gain access to one of first lumen 2 and second lumen 4, via the other of first lumen 2 and second lumen 4. For example, when access to first lumen 2 or second lumen 4 is obstructed by, e.g., a clot, occlusion, or other blockage, fistula/anastomosis 14 can be used to insert tools into the blocked lumen via another lumen that is not obstructed.

The following example will discuss an exemplary procedure when first lumen 2 is obstructed. For example, when access to first lumen 2 is obstructed, a medical practitioner may guide a device, e.g., an endoscope, bronchoscope, or the like, through second lumen 4, and may create second opening 12 in second wall 8, and first opening 10 in first wall 6. Fistula/anastomosis 14 then may be extended through second and first openings 12 and 10 to couple second and first lumens 4 and 2 to one another. Once a medical procedure is performed, any tools or devices used during that procedure may be removed. Closure device 100 then may be inserted through second lumen 4, fistula/anastomosis 14, and into first lumen 2. In one example, first covering 102 may be inserted into first lumen 2 and arranged so as to cover first opening 10. Attachment features 108 may attach to an interior of first wall 6 to prevent the flow of bodily fluid, e.g., blood, from displacing closure device 100 downstream of the installation location. Once first covering 102 is secured over opening 10, second covering 104 may be secured over second opening 12. In some examples, this step may include applying a force against the tension force of tension member 106. Once both first and second coverings 102 and 104 are positioned over respective openings, the medical practitioner may release the force applied to tension member 106 to allow tension member 106 to urge toward the closed position (the shortened configuration), thereby securing first and second coverings 102 and 104 within the body. Once secured, first and second coverings 102 and 104 may close or otherwise seal the artificial connection formed between first and second lumens 2 and 4 by fistula/anastomosis 14.

While the above procedure describes a method in which first lumen 2 was obstructed, those of ordinary skill in the art will appreciate that a similar method of forming and closing a fistula/anastomosis may be used when second lumen 4 is obstructed such that access to second lumen 4 is achieved via first lumen 2.

In some examples, closure device 100 may be used between two or more lumens that are free of obstructions. In another example, closure device 100 may be used to close a fistula/anastomosis 14 or other suitable connection formed between one organ and an adjacent organ. In some examples, closure device 100 may be used to close a percutaneous delivery or access device in a portion of the body, for example, a leg.

We claim:

1. A closure device, comprising:
a first covering configured to cover a first opening in a first body lumen;
a second covering configured to cover a second opening in a second body lumen; and
a tension member coupled to both the first opening and the second opening, the tension member being reciprocally movable between a collapsed configuration and an expanded configuration, wherein the tension member is biased toward the collapsed configuration.
